SEQUENCE LISTING (15 sequences)

SEQ ID NO: 1 - protein, 9 aa, Artificial Sequence
  DISULFID 1..6: disulfide bridge
  Oxytocin
CYIQNCPLG

SEQ ID NO: 2 - protein, 9 aa, Artificial Sequence
  MOD_RES 1: Mpr SULFATATION
  DISULFID 1..6: disulfide bridge
  Desmopressin
XYFQNCPRG

SEQ ID NO: 3 - protein, 6 aa, Artificial Sequence
  Dalargin
YAGFLR

SEQ ID NO: 4 - protein, 9 aa, Artificial Sequence
  MOD_RES 1: pGlu PYRROLIDONE CARBOXYLIC ACID
  Lecirelin
  MOD_RES 9: NHEt
EHWSYLLRP

SEQ ID NO: 5 - protein, 9 aa, Artificial Sequence
  MOD_RES 1: pGlu PYRROLIDONE CARBOXYLIC ACID
  Fertitrelin
  MOD_RES 9: NHEt
EHWSYGLRP

SEQ ID NO: 6 - protein, 12 aa, Artificial Sequence
  Terlipressin
  DISULFID 4..9: disulfide bridge
GGGCYFQNCPKG

SEQ ID NO: 7 - protein, 9 aa, Artificial Sequence
  DISULFID 1..6: disulfide bridge
  Lysipressin
CYFQNCPKG

SEQ ID NO: 8 - protein, 7 aa, Artificial Sequence
  MOD_RES 1: Ac ACETYLATION
  Effect on adhesive properties
  MOD_RES 7: Nle
RGDGGGX

SEQ ID NO: 9 - protein, 7 aa, Artificial Sequence
  MOD_RES 1: Fmoc
  Effect on adhesive properties
  MOD_RES 7: Nle
RGDGGGX

SEQ ID NO: 10 - protein, 6 aa, Artificial Sequence
  MOD_RES 1: Ac ACETYLATION
  Effect on adhesive properties
GGGRGD

SEQ ID NO: 11 - protein, 6 aa, Artificial Sequence
  MOD_RES 1: Fmoc
  Effect on adhesive properties
GGGRGD

SEQ ID NO: 12 - protein, 6 aa, Artificial Sequence
  MOD_RES 1: Ac ACETYLATION
  Effect on adhesive properties
  MOD_RES 4: Ahx
  MOD_RES 5: Ahx
  MOD_RES 6: Nle
RGDXXX

SEQ ID NO: 13 - protein, 6 aa, Artificial Sequence
  MOD_RES 1: Fmoc
  Effect on adhesive properties
  MOD_RES 4: Ahx
  MOD_RES 5: Ahx
  MOD_RES 6: Nle
RGDXXX

SEQ ID NO: 14 - protein, 5 aa, Artificial Sequence
  MOD_RES 1: Ahx
  MOD_RES 1: Ac ACETYLATION
  Effect on adhesive properties
  MOD_RES 2: Ahx
XXRGD

SEQ ID NO: 15 - protein, 5 aa, Artificial Sequence
  MOD_RES 1: Ahx
  MOD_RES 1: Fmoc
  Effect on adhesive properties
  MOD_RES 2: Ahx
XXRGD

(19) United States
(12) Patent Application Publication
(10) Pub. No.: US 2019/0328891 A1
(43) Pub. Date: Oct. 31, 2019

(54) MEDICAL PREPARATION WITH A CARRIER BASED ON HYALURONAN AND/OR DERIVATIVES THEREOF, METHOD OF PREPARATION AND USE THEREOF

(71) Applicant: CONTIPRO A.S., Dolni Dobrouc (CZ)

(72) Inventors: Sergej KAREL, Usti Nad Orlici (CZ); Martin FLEGEL, Psary - Dolni Jircany (CZ); Romana SULAKOVA, Usti Nad Orlici (CZ); Roman FRYCAK, Bohunovice (CZ); Jana SOGORKOVA, Hradek U Rokycan (CZ); Jiri BETAK, Dolni Ujezd (CZ); Josef CHMELAR, Vsetin (CZ); Vojtech ZAPOTOCKY, Slany (CZ); Vladimir VELEBNY, Zamberk (CZ)

(21) Appl. No.: 16/472,635

(22) PCT Filed: Dec. 19, 2017
(86) PCT No.: PCT/CZ2017/050061

§ 371 Date: Jun. 21, 2019

(30) Foreign Application Priority Data
Dec. 22, 2016 (CZ) PV 2016-826

(51) Int. Cl. A61K 47/61; A61K 9/00; A61K 47/62; A61K 47/69; A61K 38/08

(52) U.S. Cl. CPC A61K 47/61; A61K 9/0053; A61K 9/006; A61K 38/08; A61K 47/62; A61K 47/6953; A61K 9/0014

(57) ABSTRACT

Disclosed is a method of preparing a medical preparation with a carrier based on hyaluronan and/or its derivatives, which can be used in the field of medicine and cosmetics. The medical preparation comprises a conjugate of hyaluronic acid and/or its derivative with a medical substance, according to the general formula A-S—N, where: A is the medical substance; S is the way of linking the medical substance with the carrier; and N is the carrier based on hyaluronic acid and/or its derivatives.

### CROSS-REFERENCE TO RELATED APPLICATIONS

This application is the National Stage of International Application No. PCT/CZ2017/050061, filed on 22 Dec. 2016, which claims priority to and all advantages of CZ Application No. PV2016-826, filed on 22 Dec. 2016, the content of which is hereby incorporated by reference.

### FIELD OF THE INVENTION

The invention relates to a medical preparation with a carrier based on hyaluronan and/or its derivatives where the carrier used directly carries the active agent and is present in various forms. The invention also relates to a method of preparing, and the use of, said medical preparation.

### BACKGROUND OF THE INVENTION

Hyaluronic acid (hyaluronan, HA) is known as a non-sulphated glycosaminoglycan composed of two repeating units of D-glucuronic acid and N-acetyl-D-glucosamine, linked via β(1→4) and β(1→3) glycosidic bonds, see FIG. 1. The molecular weight of the native hyaluronic acid is in the range of 5·10⁴ to 5.10⁶ g·mol⁻¹. This significantly hydrophilic polysaccharide forms part of connective tissues, skin, synovial fluid of joints, and plays an important role in many biological processes such as organization of proteoglycans, hydration, and cells differentiation.

In view of the fact that this polymer is biocompatible, inherent to the body, and degradable, it becomes a suitable substrate for tissue engineering or as a carrier of bioactive substances (Schante C. E. et al.: Carbohyd Polym 85, 469 (2011)).

The use of hyaluronan and its derivatives with modified or improved properties in the field of medicine is quite wide. Chemical modification of hyaluronan is usually preformed in two reaction centres: on the carboxyl group and on the primary hydroxyl group. An amino group can also be used after cleavage of N-acetyl group. It is not clear which of the hydroxyl group of the saccharide unit participates in the reaction; many results indicate the preference of the primary hydroxyl (C6) of the N-acetylglucosamine unit because of the highest reactivity of this group.

The carboxyl group can be converted into an amide or an ester. Activating agents conventionally used for chemical preparation of peptides, such as carbodiimides (see, e.g. Danishefsky I. et al. Carbohyd Res 16 (1), 199 (1971); Bulpitt P. et al. J Biomed Mater Res 47 (2), 152 (1999); Foullain N. et al., Carbohyd Polym 74 (3), 333 (2008); and Oh E. et al. J Control Release 141 (1), 2 (2010)) or other agents, such as 2-chloro-1-methylpyridiniumiodide (see, e.g. Magnani A. et al. Polym Advan Technol 11 (8-12), 488 (2000)), 2-chloro-dimethoxy-1,3,5-triazine (see, e.g. Bergman K. et al. Biomacromolecules 8 (7), 2190 (2007)), carbonyldiimidazole (see, e.g. WO 2000/001733) etc., can be used for the conversion of the carboxyl group to an amide. After increasing the carboxyl reactivity through conversion into a more reactive intermediate, an amine is added to provide an amide by a condensation reaction. An ester can be formed by an alkylation reaction with alkylhalogenides (see, e.g. U.S. Pat. No. 4,851,521 and Pelletier S. et al. Carbohyd Polym 43 (4), 343 (2000)), via methyl ester synthesis by reaction with diazomethane (see, e.g. Jeanloz R. et al. J Biol Chem 186 (2), 495 (1950) and Hirano K. et al. Carbohyd Res 340 (14), 2297 (2005)), via use of an epoxide (see, e.g. Leach J. et al. Biotechnol Bioeng 82 (5), 578 (2003); Bencherif S. et al. Biomaterials 29 (12), 1973 (2008); Weng L. et al. Biomaterials 29 (14), 2153 (2008); and Prata J. et al. Biomacromolecules 11(3), 769 (2010)), etc.

The modification of a hydroxyl group leads to the formation of an ether or an ester bond. Ethers can be easily prepared by the reaction with epoxides (see, e.g. Laurent T. et al. Acta Chem Scand 18 (1), 274 (1964); Yui N. et al. J Control Release 22 (2), 105 (1992); Tomihata K. et al. Biomaterials 18 (3), 189 (1997); and WO 2000/046253), divinylsulphones (see, e.g. U.S. Pat. No. 4,582,865; Ramamurthi A. et al. J Biomed Mater Res 60 (1), 196 (2002); and Eun J. et al. J Biomed Mater Res-A 86 (3), 685 (2008)), ethylensulphides (see, e.g. Serban M. et al. Biomaterials 29 (10), 1388 (2008)), or by formation of a hemiacetal with the use of glutaraldehyde (see, e.g. Tomihata K. et al. J Polym Sci Pol Chem 35 (16), 3553 (1997) and Crescenzi V. et al. Carbohyd Polym 53 (3), 311 (2003)). Symmetric or mixed anhydrides can be used for an ester synthesis (see, e.g. CZ302856, WO/2014/082609, and Huerta-Angeles G. et al. Carbohyd Polym 111, 883 (2014)), and O-acyl-O′-alkylcarbonate may be used as an activating agent (see, e.g. WO 2010/105582). These structural modifications lead to a crosslinking reaction to form hydrogels.

Solid Phase Peptide Synthesis, SPPS, is a method where individual amino groups are gradually linked to a polymer carrier. Protecting groups (permanent or temporary) are used for all amino acids to decrease the risk of side reactions and formation of undesirable sequences. Protecting groups such as Fmoc and Boc are the most frequently used, where Fmoc protecting group can be cleaved in a basic environment and Boc protecting group can be cleaved in an acid environment. Solid phase peptide synthesis comprises repeating of the cycles of “condensation-washing-cleavage of protecting groups-washing”. A synthetized peptide remains anchored to the polymer carrier until its desired sequence is obtained. Then a cleavage from the carrier and purification are performed.

Nowadays there is an effort to use carriers that can be biocompatible and optionally biodegradable. The carriers based on polysaccharides from alginate, agar, chitin, hyaluronan or cotton have been described. So far, the peptides have always been cleaved from the carriers. The beneficial biological properties, that can mutually positively influence the biological properties of the peptide (see, e.g. Eichler J. et al.: Cotton-carrier for solid phase peptide synthesis (1991) and WO 2012/101612) synthetized or anchored on the carrier, have not been used.

Alginates are the most common carriers used for the reaction of peptides and amino acids with polysaccharides. Alginate is a linear negatively charged polysaccharide consisting of repeating monomer units of β-D-mannuric acid and α-L-guluronic acid linked via (1-4) O-glykosidic bonds, as it is shown in the structural formula of the alginate in Figure. 2.

The use of alginate as a drug form for pharmaceuticals was described; however the processes were most often used for non-covalent anchoring of these drugs in a matrix or a gel. The alginate gel has been used also for analytical procedures of capturing the high molecular peptides, mainly enzymes, cell organelles, or the whole cells (see, e.g. Palmieri G. et al. J Chromatogr B 664, 127 (1995) and Morgan S. M. et al. Int J Pharm 122, 121 (1995)).

Covalent bonding of peptides and drugs enables their use as sophisticated dosage systems enhancing pharmacokinetics and bioavailability which enhances the clinical potential of a drug. The conjugate alginate-peptide is formed by an amidic bond between the carboxyl group of the alginate and the amino group of the peptide (see, e.g. Palmieri (1995); Morgan (1995), and Hashimoto T. et al. Biomaterials 25, 1407 (2004)).

The alginates with covalently bound peptides can be used in treatment of an injury. Wound healing includes a tissue reaction to an injury. It is a complicated biological process comprising chemotaxis, cell proliferation, production of extracellular proteins, neovascularization etc. One of the possibilities of wound healing treatment is influencing the healing process by natural stimulation agents such as growth factor (see, e.g. Hashimoto (2004)).

Materials based on alginates have been prepared, which were modified by a peptide, and their effectivity in the wound healing process was studied on the injured skin in vivo. Rabbits treated with an alginate bandage containing a hybrid peptide Ser-Ile-Arg-Val-X-Val-X-Pro-Gly (where X=Ala or Gly) showed significantly higher epithelization and larger volume of regenerated tissue in comparison with other peptides Ser-Ile-Lys-Val-Ala-Val and Val-Pro-Val-Ala-Pro-Gly that were also anchored on the alginate bandage (see, e.g. Hashimoto (2004)).

Cellulose in the form of paper was also used for the preparation of peptides. From a chemical point of view, cellulose is a linear polysaccharide consisting of repetitive monomer units of d-glucose linked via β(1→4) O-glycosidic bonds, according to the structural formula shown in FIG. 3. Peptides can only be obtained with low yield, which is not desirable. Paper also has the disadvantage of low mechanical stability (see, e.g. Eichler (1991), and Frank R. et al. Tetrahedron 44, 6031 (1988)).

Cotton is the purest form of cellulose, excepting microbial cellulose, and can be obtained in various forms and shapes; the possibility of the use of cotton as the carrier for SPPS has been studied. In this case, the anchoring of the peptide was performed by means of an ester bond between the hydroxyl group of cotton and the carboxyl group of the peptide. The synthesis was most often performed in DIC/HOBt/DMAP in DMF (see, e.g. Eichler et al. “Cotton-carrier for solid phase peptide synthesis” (1991); Pept Res 4 (5), 296 (1991); and “Innovation and Perspectives in Solid Phase Synthesis,” Birmingham, UK: SPCC, 337-343 (1990)).

Nowadays, carriers based on hyaluronan in the form of fibres (see, e.g. WO 2012/089179, WO 2013/167098, WO 2014/082610, and WO 2014/082611), thin films (see, e.g. WO 2016/141903 and Foglarová M. et al. Carbohydr Polym 144, 68 (2016)), or nonwoven fabrics (see, e.g. WO/2013/167098) are available. The use of these perspective forms could remove some of the described drawbacks of the polysaccharide carriers used so far. However, there is still a lack of suitable carriers of pharmaceutical drugs.

### SUMMARY OF THE INVENTION

The new method described herein has the advantage of the use of a carrier based on hyaluronan in the form of fibres, thin films, or nonwoven fabrics for solid-phase synthesis. This carrier does not change during the reaction, i.e., it remains in the form of fibres, thin films, or nonwoven fabrics. Thus, it is a solid-phase synthesis where the peptide is constructed by individual amino acids or it is linked, as a whole, to the hyaluronan material. So far, hyaluronan has been used for the modification in solution, i.e., hyaluronan was dissolved and the reaction performed.

Then the derivative was used for the preparation of the material. The difference between liquid-phase and solid-phase synthesis is that in case of solution synthesis and subsequent spinning, the peptide is also present inside the fibre, so it is less available. The solid-phase synthesis saves costs, and the peptide being on the surface only spares from the optimization of the spinning processes which are different for individual derivatives.

In particular, the abovementioned drawbacks are solved by a method of preparing a medical preparation with a carrier based on hyaluronan and/or derivatives thereof, and by the medical preparation with the carrier based on hyaluronan and derivatives thereof, as described herein and defined in the appended claims.

The medical preparation comprises a conjugate of hyaluronic acid and derivatives thereof with a medical substance, according to the general formula (X):

A-S—N (X),

or the formula:

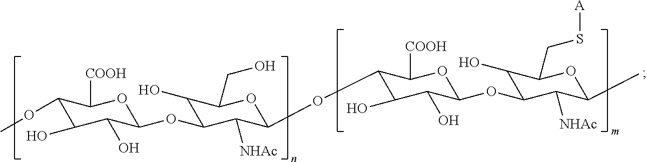

where

each A is the medical substance selected from the group comprising amino acids and peptides,

each S is a method of linking (i.e., a linker/linking group, e.g. a divalent linking group) of the medical substance with the carrier and comprises —O— or —NH—,
N is the carrier based on hyaluronic acid and/or derivatives thereof,
subscript m is 10 to 1250, and
subscript n is 100 to 12500.

In some embodiments, the carrier of the medical preparation, alternatively the medical preparation itself, is water-insoluble and biodegradable.

In certain embodiments, the medical preparation is prepared without the carrier being dissolved. In other words, the carrier remains insoluble during the whole preparation process, i.e., in solid phase, wherein at the same time it maintains its biodegradability.

In particular embodiments, the medical preparation comprises the carrier in the form of an endless fibre, thread, fabrics, thin film, staple fibre and/or nonwoven textile.

In specific embodiments, the medical preparation comprises the medical substance bonded onto the carrier in the position 6 of the glucosamine part of the conjugate of hyaluronic acid and/or its derivatives according to the general formula X.

In some embodiments, the medical substance is anchored to hyaluronic acid and/or its derivatives via an ester bond (e.g. according to Scheme 1 below) or by reductive amination (e.g. according to Scheme 2 below).

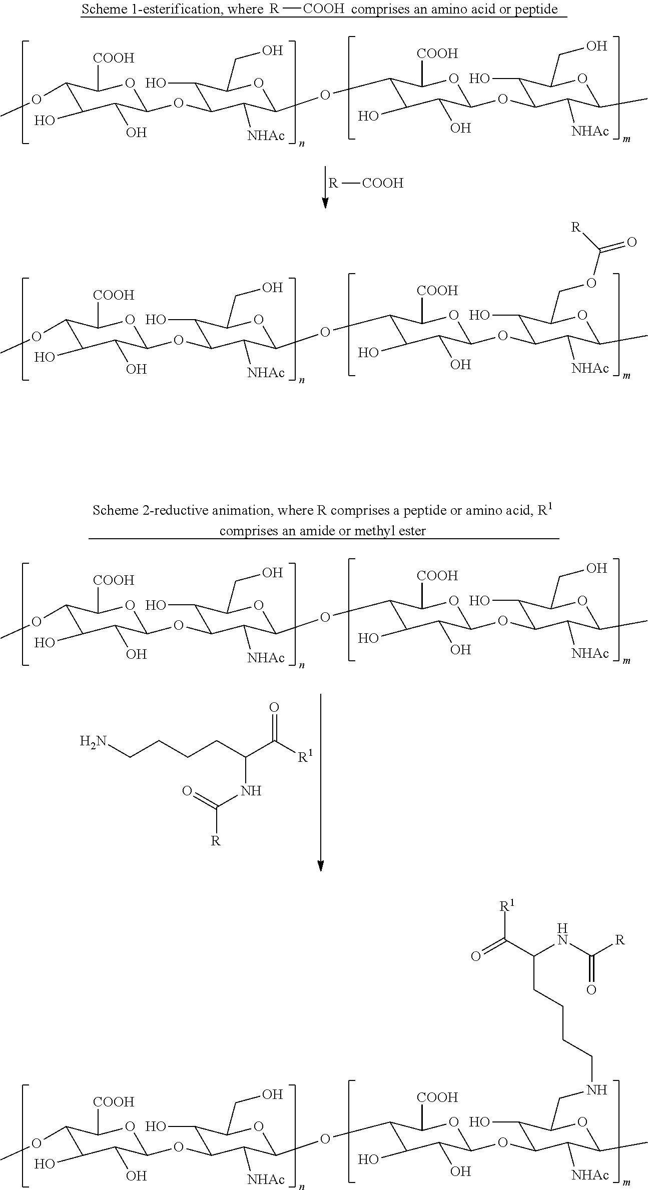

In some embodiments, the carrier comprises, alternatively is, hyaluronic acid, palmitoyl hyaluronan, or formyl hyaluronan. In these or other embodiments, the carrier has a molecular weight in the range of from 1.5×10⁴ to 2.5×10⁶ g·mol⁻¹.

As introduced above, this disclosure also provides a method of preparing the medical preparation with a carrier based on hyaluronan and/or its derivatives where the carboxyl group of an amino acid or peptide in the medical substance A, which comprises an N-terminal protecting group (i.e., a terminal N-protecting group), is activated with a condensation agent in an aprotic polar solvent to form a reactive intermediate I, which reacts with the carrier in the presence of an organic base and a catalyst to form a conjugate of hyaluronic acid and/or derivatives thereof with the medical substance. Thereafter, the terminal N-protecting group of the medical substance is cleaved off in a basic medium. Typically, the basic medium is selected from a group consisting of, 20% piperidine in N,N-dimethylformamide, 2% 1,8-diazabicyclo[5.4.0]undec-7-ene in N,N-dimethylformamide, and 30% tert-butylamine in N,N-dimethylformamide. In specific embodiments, the basic medium comprises, alternatively is, 20% piperidine in N,N-dimethylformamide.

The medical substance A is typically selected from the group consisting of amino acids and peptides (i.e., the medical substance A comprises an amino acid and/or a peptide). The medical substance A is linked to the carrier directly, or via a linear linker based on a peptide formed by hydrophobic amino acids Xaa, such as Xaa-Ahx-Ahx-Xaa.

In some embodiments, the method comprises forming the conjugate of hyaluronic acid and/or its derivatives at a temperature of from 20° C. to 40° C., such as at a temperature of from 22° C. to 25° C., for a time of from 2 to 48 hours, such as for 24 hours.

The condensation agent utilized in the method is typically selected from the group of 4-nitrophenol, N-hydroxy succinimide, 2,4,5-trichlorophenol, 2,3,4,5,6-pentafluorophenol, 2,3,4,5,6-pentachlorophenol, N,N′-diisopropyl carbodiimide, N,N′-dicyclohexyl carbodiimide, l-[bis(dimethylamino) methylen]-1H-1,2,3-triazolo[4,5-b] pyridinium3-oxide hexafluoro phosphate, 1-ethyl-3-(3-dimethylaminopropyl) carbodiimide, ethylchloroformiate, 2-(1H-benzotriazole-1-yl)-1,1,3,3-tetramethyl uronium hexafluoro phosphate, benzotriazol-1-yl-oxy-tris(dimethylamino)-phosphonium hexafluoro phosphate, O-(benzotriazol-1-yl)-N,N,N′,N′-tetramethyl uronium tetrafluoro borate, 4-triethylamino-dicyclohexyl karbodiimide p-toluensulphonate, and propyl phosphonic acid anhydride. In specific embodiments, the condensation agent comprises, alternatively is, N,N′-diisopropyl carbodiimide.

The polar aprotic solvent utilized in the method is typically selected from the group comprising N,N-dimethyl formamide, dimethyl sulphoxide, N-methyl-2-pyrrolidone, acetonitrile, dichloromethane, tetrahydrofuran, and 1,4-dioxane. In certain embodiments, the polar aprotic solvent comprises, alternatively is, N,N-dimethyl formamide.

The organic base utilized in the method is typically selected from the group comprising triethylamine, pyridine, morpholine, N-methyl morpholine, N,N′-diisopropyl ethylamine, and imidazole.

The catalyst utilized in the method is typically selected from the group comprising ethyl (hydroxyimino) cyanoacetate, hydroxyl benzo triazole, N,N-dimethyl amino pyridine or 1-hydroxy-7-azabenzo triazole. In some embodiments, the catalyst comprises ethyl (hydroxyimino) cyanoacetate and/or N,N-dimethyl amino pyridine.

The method according to the invention is also characterized in that the amount of the amino acid or peptide used, i.e., in/as the medical substance, corresponds to from 1 to 5 equivalents, alternatively 3 equivalents, based on the dimer of hyaluronic acid and/or its derivative(s); the amount of the activating (condensation) agent used corresponds to from 0.1 to 5 equivalents, alternatively 3 equivalents, based on the dimer of hyaluronic acid and/or its derivative(s); the amount of the base used corresponds to up to 10 equivalents, based on the dimer of hyaluronic acid and/or its derivative(s); and the amount of the catalyst used corresponds to from 0.1 to 5 equivalents, alternatively 3 equivalents of ethyl (hydroxyimino) cyanoacetate and 0.3 equivalents of N,N-dimethyl amino pyridine, per dimer of hyaluronic acid and/or its derivative(s). In particular embodiment of the invention, the molar ratio of the medical substance, condensation agent, catalyst, and hyaluronic acid dimer is 1:1:1:1, respectively.

The medical preparation in the form of the conjugate, according to the invention is for medical use as a dosage form of an active peptide or amino acid, intended for dermal, sublingual, oral, buccal, or local administration into an open wound.

For example, in certain embodiments the medical preparation according to the invention is for dermal administration and can contain, for example, the peptide Dalargin as the medical substance. In some embodiments, the medical preparation according to the invention is for buccal or sublingual administration and can contain, for example, a peptide selected from the group of Desmopressin, Lysipressin, and Glypressin, as the medical substance. Likewise, the medical preparation according to the invention for buccal administration can contain, for example, a peptide selected from the group of antiviral drugs and adjuvants, and releasing factor for luteinizing and/or follicle-stimulating hormone. In some embodiments, the medical preparation according to the invention is for direct administration to an open wound and can contain, as the medical substance, for example, a peptide selected from the group of Glypressin, Dalargin, and AdDP (e.g. as an immunostimulant).

Thus, one aspect of the invention according to certain embodiments is the new compounds of the general formula (X):

A-S—N (X),

where the solid carrier N is made of hyaluronan and/or its derivatives in the form of an endless fibre, thread, fabric, thin film, staple fibre and nonwoven fabric comprising the medical substance A linked by means of linkage S, and the method of the preparing this complex system by the procedure in solid phase, or by bonding the medical substance into a complex system, as described herein and illustrated in the examples below.

In certain embodiments, the method of linking the medical substance with the carrier is performed via esterification (e.g. by formation of an ester bond) or by reductive amination. The carrier is a biocompatible and biodegradable solid carrier characterized in that it does not need to be cleaved from the medical substance during its various, mainly medical (i.e., bioactive), activities, and that it can further act as a modern non-invasive dosage form facilitating the penetration of biologically active peptides or amino acids bound on said biocompatible carrier through mucosa and skin, and thus it serves as a complex and suitable material used for medical applications in human and veterinary medicine. The medical preparation according to the invention acts as a whole during the biological activity and the medical substance can be released slowly, in a prolonged manner from the carrier, which is subsequently eliminated naturally and intrinsically. The carrier can also affect the total effect of the medical substance bonded thereon and facilitate its use.

Table 1 presents peptides and their medical effect and the method of their administration, which can be utilized in the medical preparation and related methods of this disclosure. More specifically, it shows that the medical preparation according to the invention for dermal administration comprises peptide Dalagrin as the medical substance having a healing effect, or Oxytocin having hormonal effect. For oral, buccal or sublingual administration, the medical preparation according to the invention comprises the peptide Desmopressin showing an antidiuretic effect, influence on blood coagulation by increasing the VIII factor, Lysipressin having presoric effect mainly in dental surgery, or Glypressin (Terlipressin) with a prolonged pressoric effect suitable to stop bleeding. For buccal administration, the medical preparation according to the invention comprises a peptide as the medical substance, used as antiviral agent and adjuvant, for example AdDP, or releasing factor for luteinisation and follicle stimulating hormones (Fertirelin, Lecirelin). The medical preparation according to the invention for local administration into an open wound comprises peptide as the medical substance used to stop bleeding (Terlipressin), and Dalargin for accelerating the wound healing, optionally AdDP for local stimulation of the immune system.

Table of peptides

| | | Sequence | |
|---|---|---|---|
| Peptide name | Peptide sequence | no. | Possible pharmacological effect |
| Oxytocin | Cys-Tyr-Ile-Gln-Asn-Cys-Pro-Leu-Gly-NH₂ | SEQ ID 1 | Affects milk-ejecting activity |
| | (disulphide bridge Cys¹-Cys⁶) | | |
| Desmopressin | Mpr-Tyr-Phe-Gln-Asn-Cys-Pro-D-Arg-Gly- | SEQ ID 2 | Affects diuresis and bleeding |
| | NH₂ | | conditions |
| | (disulphide bridge Mpr¹-Cys⁶) | | |
| Dalargin | H-Tyr-D-Ala-Gly-Phe-Leu-Arg-OH | SEQ ID 3 | Healing effects |
| Lecirelin | pG1u-His-Trp-Ser-Tyr-D-tertLeu-Leu-Arg- | SEQ ID 4 | Affects releasing factor for |
| | Pro-NHEt | | luteinisation and follicle |
| | | | stimulating hormones |
| Fertirelin | pGlu-His-Trp-Ser-Tyr-Gly-Leu-Arg-Pro- | SEQ ID 5 | Affects the releasing factor |
| | NHEt | | for luteinisation and follicle |
| | | | stimulating hormones |
| Terlipressin | Gly-Gly-Gly-Cys-Tyr-Phe-Gln-Asn-Cys- | SEQ ID 6 | Affects blood pressure |
| (Glypressin) | Pro-Lys-Gly-NH₂ | | |
| | (disulphide bridge Cys¹-Cys⁶) | | |
| Lysipressin | Cys-Tyr-Phe-Gln-Asn-Cys-Pro-Lys-Gly- | SEQ ID 7 | Affects blood pressure |
| (8-Lys- | NH₂ | | |
| vasopressin) | (disulphide bridge Cys¹-Cys⁶) | | |
| AdDP | Ala-igln-NH-Ad | | Antiviral agent and adjuvant |
| | Ac-Arg-Gly-Asp-Gly-Gly-Gly-Nle-OH | SEQ ID 8 | Affects adhesive properties |
| | Fmoc-Arg-Gly-Asp-Gly-Gly-Gly-Nle-OH | SEQ ID 9 | (in vitro) |
| | Ac-Gly-Gly-Gly-Arg-Gly-Asp-OH | SEQ ID 10 | |
| | Fmoc-Gly-Gly-Gly-Arg-Gly-Asp-OH | SEQ ID 11 | |
| | Ac-Arg-Gly-Asp-Ahx-Ahx-Nle-OH | SEQ ID 12 | |
| | Fmoc-Arg-Gly-Asp-Ahx-Ahx-Nle-OH | SEQ ID 13 | |
| | Ac-Ahx-Ahx-Arg-Gly-Asp-OH | SEQ ID 14 | |
| | Fmoc-Ahx-Ahx-Arg-Gly-Asp-OH | SEQ ID 15 | |

### BRIEF DESCRIPTION OF THE DRAWINGS

The enclosed FIG. 1 shows hyaluronan where R=H+ or Na+ and n is equal to 100 to 12500.

FIG. 2 shows the structural formula of alginate.

FIG. 3 shows the structural formula of cellulose.

FIG. 4 shows the structural formula of the medical preparation according to the invention, where A is the medical substance, S is the way of linking the medical substance with a carrier, N is the carrier based on hyaluronic acid and its derivatives, subscript m is from 10 to 1250, and subscript n is from 100 to 12500.

FIG. 5 shows the structural formula of hyaluronic acid or hyaluronic acid derivative with the bound peptide, where HYA is hyaluronic acid or its derivative, and the peptide comprises 7 lysine units arranged in a dendrimer structure.

FIG. 6 shows DiI stained NHDF cells adhered to a fibre of native HYA comprising RGD motives. The images were obtained by means of fluorescent microscope after 48 hours of cultivation.

FIG. 7 shows changes in the presence of non-adhered DiI stained NHDF cells population. The images were obtained by means of fluorescent microscope after 1, 2, 4, and 7 days of cultivation.

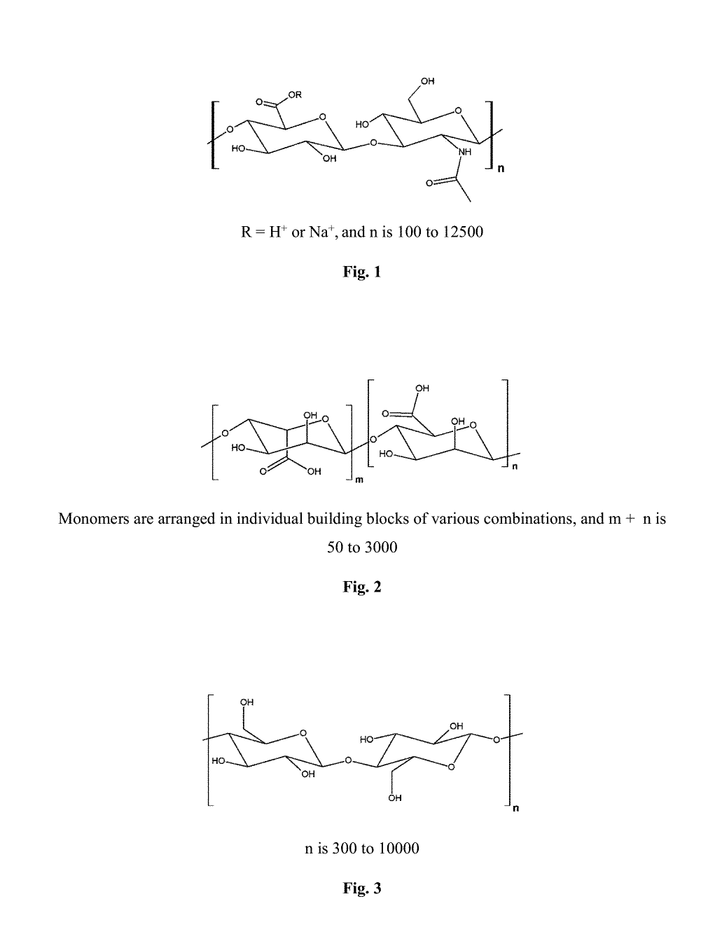

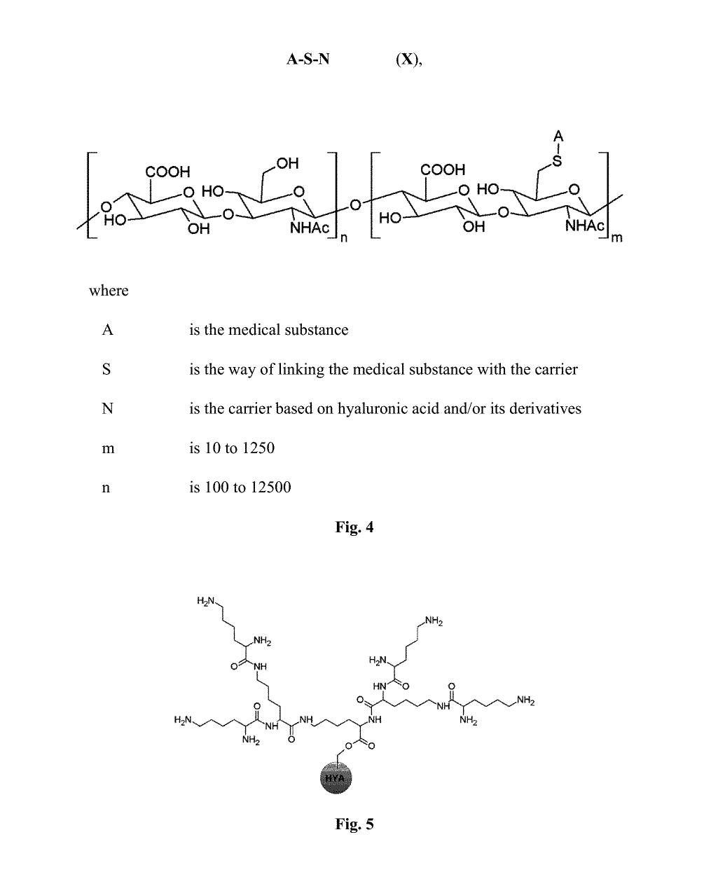

### DESCRIPTION OF THE EMBODIMENTS

The conjugate of peptide with the material based on hyaluronan according to the invention was successfully prepared and tested by the authors in the applicant's facilities—Contipro a.s., Dolní Dobruč, CZ.

### LIST OF ABBREVIATIONS

AdDP 1-adamantylamide-(L)-alanyl-(D)-isoglutamine
Ahx aminohexanoic acid
Boc tert-butyloxycarbonyl
Castro reagent benzotriazol-1-yl-oxy-tris(dimethylamino)phosphonium hexafluoro phosphate
DBU 1,8-diazabicyclo[5.4.0]undec-7-ene
DCC N,N′-dicyclohexylcarbodiimide
DCM dichloromethane
DEE diethylether
DIC N,N′-diisopropylcarbodiimide
DiI 1,1′-dioctadecyl-3,3,3′,3′-tetramethyl indocarbocyanine perchlorate
DIPEA N,N′-diisopropyl ethylamine
DMAP N,N-dimethyl aminopyridine
DMF N,N-dimethyl formamide
EDC 1-ethyl-3-(3-dimethyl aminopropyl) carbodiimide
Fmoc fluorenyl methyloxy carbonyl
HATU 1-[bis(dimethylamino) methylen]-1H-1,2,3-triazolo[4,5-b] pyridinium 3-oxide hexafluoro phosphate
HBTU 2-(1H-benzotriazole-1-yl)-1,1,3,3-tetramethyl uronium hexafluoro phosphate
HOAt 1-hydroxy-7-azabenzo triazole
HOBt N-hydroxy benztriazole
HOSu N-hydroxy succinimide
IPA isopropyl alcohol
OxymaPure ethyl (hydroxyimino) cyanoacetate
picBH₃ picolin borane complex
T3P propyl phosphonic acid anhydride
TBTU O-(benzotriazole-1-yl)-N,N,N,N′-tetramethyl uronium tetrafluoro borate
WSCD 4-triethylamino dicyclohexyl carbodiimide p-toluen sulphonate

The fibre from the native HYA and nonwoven fabric from the native HYA were prepared according to the process described in the patent WO/2013/167098. The fibre from palmitoyl HYA was prepared according to the process described in the patent WO/2014/082611.

In the following examples, the term “equivalent (eq.)” relates to a repetitive unit of the respective form of hyaluronan. The percentage is per volume, if not stated otherwise.

The molecular weight of hyaluronan starting forms is the weight average molecular weight determined by the SEC-MALLS method.

In the examples, for the purposes of subsequent analysis only, norleucin, as the non-essential amino acid, was bound first to easily determine proportional occurrence of amino acids. However, norleucin bonding is not the necessary condition and is not intended to limit the scope of the invention.

### Example 1

### Anchoring of Fmoc-Nle-OH to the Fibre of Native HYA

1 m of the fibre of native HYA (12 mg, 0.03 mmol, Mw=3.10 g·mol⁻¹) was placed into a 2 ml syringe reactor equipped with a frit; the fibre was repeatedly washed with DMF. The solution of 3 eq. of Fmoc-Nle-OH, 3 eq. of OxymaPure and 0.3 eq. of DMAP in 0.5 ml anhydrous DMF was prepared outside the reactor; after dissolving all of the components 3 eq. of DIC were added to activate the carboxyl group of an amino acid. This solution was transferred into the reactor to the fibre. The reaction proceeded at the temperature of 18 to 23° C. for the next 20 hours and it was terminated by filtering the reaction solution off. The fibre was then washed with 3×1.5 ml DMF, 3×1.5 ml DCM, 3×1.5 ml IPA, 3×1.5 ml DCM, 3×1.5 ml DEE.

The yield of the condensation reaction was determined by Fmoc-release test as the substitution S=0.01862 mmol/g.

### Example 2

### Preparation of H-Nle-O-HYA

1 m of the fibre of Fmoc-Nle-O-HYA prepared according to the Example 1 was placed into a 2 ml syringe reactor equipped with a frit; the fibre was repeatedly washed with DMF. Fmoc protecting group was cleaved off by means of adding 1.5 ml of 20% solution of piperidine in DMF into the reactor to the fibre. The reaction proceeded for 5 min at the temperature of 18 to 23° C. The reaction was repeated while extending the time of cleavage to 20 min and then it was terminated by filtering the reaction solution off. The fibre was then washed with 3×1.5 ml of DMF, 3×1.5 ml of DCM, 3×1.5 ml of IPA, 3×1.5 ml of DCM, 3×1.5 ml of DEE.

The reaction is quantitative. Releasing of the amino group was confirmed by performing the ninhydrin confirmation reaction (Kaiser test).

### Example 3

### Anchoring of Fmoc-Nle-OH to the Fibre of Native HYA

1 m of fibre of native HYA (12 mg, 0.03 mmol, Mw=3.10⁵ g·mol⁻¹) was placed into a 2 ml syringe reactor equipped with a frit, the fibre was repeatedly washed with THF. A solution of 3 eq. of Fmoc-Nle-OH, 3 eq. of OxymaPure and 0.3 eq. of DMAP in 0.5 ml THF; after dissolving all the components 3 eq. of DIC were added to activate the carboxyl group of the amino acid. This solution was transferred into the reactor with the fibre. The reaction proceeded at the temperature of 18 to 23° C. for the next 20 hours and it was terminated by filtering the reaction solution off. The fibre was then washed with 3×1.5 ml of THF, 3×1.5 ml of DCM, 3×1.5 ml of IPA, 31.5 ml of DCM, 31.5 ml of DEE. The yield of the condensation reaction was determined by Fmoc-release test as the substitution S0,01759 mmol/g.

### Example 4

### Anchoring of Fmoc-Nle-OH to the Fibre of Native HYA

1 m of the fibre of native HYA (12 mg, 0.03 mmol, Mw=3.10⁵ g·mol⁻¹) was placed into a 2 ml syringe reactor equipped with a frit; the fibre was repeatedly washed with DMF. A solution of 1 eq. of Fmoc-Nle-OH, 1 eq. of OxymaPure, and 0.3 eq. of DMAP in 0.5 ml of anhydrous DMF was prepared outside the reactor; after dissolving all of the components 1 eq. of DIC was added to activate the carboxyl group of the amino acid. This solution was transferred into the reactor to the fibre. The reaction proceeded at the temperature of 18 to 23° ° C. for the next 20 hours and it was terminated by filtering the reaction solution off. The fibre was then washed with 3×1.5 ml of DMF, 3×1.5 ml of DCM, 3×1.5 ml of IPA, 3×1.5 ml of DCM, 3×1.5 ml of DEE.

The yield of the condensation reaction was determined by Fmoc-release test as the substitution S=0.01804 mmol/g.

### Example 5

### Preparation of Fmoc-[Lys(Boc)]₄-Nle-O-HYA

1 m of H-Nle-O-HYA prepared according to the Example 2 was placed into a 2 ml syringe reactor equipped with a frit; the fibre was repeatedly washed with DMF. The solution of 3 eq. of Fmoc-Lys(Boc)-OH, and 3 eq. of OxymaPure in 0.5 ml of anhydrous DMF was prepared outside the reactor; after dissolving all of the components 3 eq. of DIC were added to activate the carboxyl group of the amino acid. This solution was transferred into the reactor to the fibre. The reaction proceeded at the temperature of 18 to 23° C. for the next 20 hours and it was terminated by filtering the reaction solution off. The fibre was then washed with 3×1.5 ml of DMF, 3×1.5 ml of DCM, 3×1.5 ml of IPA, 3×1.5 ml of DCM, 3×1.5 ml of DMF. The Fmoc protecting group was cleaved off by means of adding 1.5 ml of 20% solution of piperidine in DMF into the reactor with the fibre. The reaction proceeded for 5 min at the temperature of 18 to 23° C. The reaction was repeated while extending the cleavage time to 20 min and then it was terminated by filtering the reaction solution off. Then the fibre was washed with 3×1.5 ml of DMF, 3×1.5 ml of DCM, 3×1.5 ml of IPA, 3×1.5 ml of DCM, 3×1.5 ml of DMF. This procedure was repeated 4 times.

The yield of the condensation reaction was determined by Fmoc-release test as the substitution in mmol/g. The product composition (nfound) was confirmed by amino acids analysis as presented in Table 2. It is a serial synthesis performed by a step-by-step method; however, this procedure was repeated for all said derivatives. The individual rows show the composition of the prepared conjugate after finishing said cycle; i.e., after finishing the procedure described in Example 3 the product having the composition described in the first row is obtained. Then the procedure is repeated, and the product described in the second row is obtained, etc.

TABLE 2

| | S | n_{calculated} | n_{found} |
|---|---|---|---|
| Compound | [mmol/g] | [nmol] | [nmol] |
| H-Lys(Boc)-Nle-O-HYA | 0.01839 | 0.290 | 0.295 |
| H-Lys(Boc)-Lys(Boc)-Nle-O-HYA | 0.01863 | 0.576 | 0.584 |
| H-Lys(Boc)-Lys(Boc)-Lys(Boc)- | 0.01888 | 0.870 | 0.876 |
| Nle-O-HYA | | | |
| H-Lys(Boc)-Lys(Boc)-Lys(Boc)- | 0.01856 | 1.189 | 1.194 |
| Lys(Boc)-Nle-O-HYA | | | |

### Example 6

### Preparation of a Peptide Comprising 7 Lysine Units Arranged in a Dendrimer Structure—Use of a Native HYA Fibre

1 m of H-Nle-O-HYA prepared according to the Example 2 was placed into a 2 ml syringe reactor equipped with a frit; the fibre was repeatedly washed with DMF. The solution of 3 eq. of Fmoc-Lys(Fmoc)-OH, and 3 eq. of OxymaPure in 0.5 ml anhydrous DMF was prepared outside the reactor; after dissolving all the components 3 eq. of DIC were added to activate the carboxyl group of the amino acid. This solution was transferred into the reactor with the fibre. The reaction proceeded at the temperature of 18 to 23° C. for the next 20 hours. The reaction was terminated by filtering the reaction solution off. The fibre was then washed with 3×1.5 ml of DMF, 3×1.5 ml of DCM, 3×1.5 ml of IPA, 3×1.5 ml of DCM, 3×1.5 ml of DMF. The Fmoc protecting group was cleaved off by means of adding 1.5 ml of 20% solution of piperidine in DMF into the reactor with the fibre. The reaction proceeded for 5 min at the temperature of 18 to 23° C. The reaction was repeated while extending the cleavage time to 20 min. The reaction was terminated by filtering the reaction solution off. The fibre was then washed with 3×1.5 ml of DMF, 3×1.5 ml of DCM, 3×1.5 ml of IPA, 3×1.5 ml of DCM, 3×1.5 ml of DMF. This procedure was repeated 2 times.

The reaction was monitored by the Fmoc-release test. The yield was determined by the Fmoc-release test. The composition of the product, shown in FIG. 5, was confirmed by an amino acid analysis, as presented in Table 3. It is a serial synthesis performed by step-by-step method; however, this procedure was repeated for all said derivatives. The individual rows show the composition of the prepared conjugate after finishing said cycle; i.e., after finishing the procedure described in Example 3 the product having the composition described in the first row is obtained. Then the procedure is repeated, and the product described in the second row is obtained, etc.

TABLE 3

| | S | n_{calculated} | n_{found} |
|---|---|---|---|
| Compound | [mmol/g] | [nmol] | [nmol] |
| H-Lys-Nle-O-HYA | 0.03381 | 0.25 | 0.262 |
| H-Lys₃-Nle-O-HYA | 0.07169 | 0.77 | 0.799 |
| H-Lys₇-Nle-O-HYA | 0.14211 | 1.84 | 1.876 |

### Example 7

### Preparation of a Peptide Comprising 7 Alanine Units on the Fibre of Native HYA

1 m of H-Nle-O-HYA prepared according to the Example 2 was placed into a 2 ml syringe reactor equipped with a frit; the fibre was repeatedly washed with DMF. A solution of 3 eq. of Fmoc-Lys(Boc)-OH, and 3 eq. of OxymaPure in 0.5 ml of anhydrous DMF was prepared outside the reactor; after dissolving all of the components 3 eq. of DIC were added to activate the carboxyl group of the amino acid. This solution was transferred into the reactor to the fibre. The reaction proceeded at the temperature of 18 to 23° C. for the next 20 hours. The reaction was terminated by filtering the reaction solution off. Then the fibre was washed with 3×1.5 ml of DMF, 3×1.5 ml of DCM, 3×1.5 ml of IPA, 3×1.5 ml of DCM, 3×1.5 ml of DMF. Cleavage of Fmoc protecting group was performed by adding 1.5 ml of 20% solution of piperidine in DMF into the reactor to the fibre. The reaction proceeded for 5 min at the temperature of 18 to 23° C. The reaction was repeated while extending the cleavage time to 20 min and it was terminated by filtering the reaction solution off. The fibre was then washed with 3×1.5 ml of DMF, 3×1.5 ml of DCM, 3×1.5 ml of IPA, 3×1.5 ml of DCM, 3×1.5 ml of DMF. This procedure was repeated 7 times for Fmoc-Ala-OH.

The yield of the condensation reaction was determined by Fmoc-release test as the substitution in mmol/g. The product composition was confirmed by an amino acids analysis as the proportional occurrence of individual amino acids. The purity of the material was determined by means of MS-HPLC after degradation of the material. The results of the individual analysis are shown in the following Table 4:

TABLE 4

| | S | Amino acid | | | Purity |
|---|---|---|---|---|---|
| Compound | [mmol/g] | Ala | Lys | Nle | [%] |
| H-(Ala)₇-Lys(Boc)- | 0.01798 | 7.28 | 1.03 | 1 | 86 |
| Nle-O-HYA | | | | | |

### Example 8

### Anchoring of Fmoc-Nle-OH to the Fibre of Palmitoyl HYA

1 m of the fibre from palmitoyl HYA (15 mg, 0.03 mmol, Mw=3.10⁵ g·mol-) was placed into a 2 ml syringe reactor equipped with a frit; the fibre was repeatedly washed with DMF. A solution of 3 eq. of Fmoc-Nle-OH, and 3 eq. of OxymaPure and 0.3 eq. of DMAP in 0.5 ml anhydrous DMF was prepared outside the reactor; after dissolving all of the components, 3 eq. of DIC were added to activate the carboxyl group of the amino acid. This solution was transferred into the reactor to the fibre. The reaction proceeded at the temperature of 18 to 23° C. for the next 20 hours and it was terminated by filtering the reaction solution off. Then the fibre was washed with 3×1.5 ml of DMF, 3×1.5 ml of DCM, 3×1.5 ml of IPA, 3×1.5 ml of DCM, 3×1.5 ml of DMF.

The yield of the condensation reaction was determined by Fmoc-release test as the substitution S=0.01029 mmol/g.

### Example 9

### Preparation of H-Nle-O-Palmitoyl HYA

1 m of Fmoc-Nle-O-palmitoylHYA prepared according to Example 8 was placed into a 2 ml syringe reactor equipped with a frit; the fibre was repeatedly washed with DMF. Fmoc protecting group was cleaved off by means of adding 1.5 ml of 20% solution of piperidine in DMF into the reactor to the fibre. The reaction proceeded at the temperature of 18 to 23° C. for 5 min. The reaction was repeated while extending the time of cleavage to 20 min and then it was terminated by filtering the reaction solution off. Then the fibre was washed with 3×1.5 ml of DMF, 3×1.5 ml of DCM, 3×1.5 ml of IPA, 3×1.5 ml of DCM, 3×1.5 ml of DEE.

The reaction is quantitative. Releasing of the amino group was confirmed by performing the ninhydrin confirmation reaction (Kaiser test).

### Example 10

### Preparation of Fmoc-[Lys(Boc)]4-Nle-O-palmitoylHYA

1 m of H-Nle-O-palmitoylHYA prepared according to the Example 9 was placed into a 2 ml syringe reactor equipped with a frit; the fibre was repeatedly washed with DMF. A solution of 3 eq. of Fmoc-Lys(Boc)-OH, and 3 eq. of OxymaPure in 0.5 ml anhydrous DMF was prepared outside the reactor; after dissolving all of the components 3 eq. of DIC were added to activate the carboxyl group of the amino acid. This solution was transferred into the reactor to the fibre. The reaction proceeded at the temperature of 18 to 23° C. for the next 20 hours and it was terminated by filtering the reaction solution off. The fibre was then washed with 3×1.5 ml of DMF, 3×1.5 ml of DCM, 3×1.5 ml of IPA, 3×1.5 ml of DCM, 3×1.5 ml of DMF. Cleavage of Fmoc protecting group was performed by adding 1.5 ml of 20% solution of piperidine in DMF into the reactor to the fibre. The reaction proceeded for 5 min at the temperature of 18 to 23° C. The reaction was repeated while extending the cleavage time to 20 min and then it was terminated by filtering the reaction solution off. The fibre was then washed with 3×1.5 ml of DMF, 3×1.5 ml of DCM, 3×1.5 ml of IPA, 3×1.5 ml of DCM, 3×1.5 ml of DMF. This procedure was repeated 4 times.

The yield of the condensation reaction was determined by Fmoc-release test as the substitution in mmol/g. The composition of the product (nfound) was confirmed by amino acid analysis, as presented in Table 5 below. It is a serial synthesis performed by the step-by-step method; however, this procedure was repeated for all the derivatives. The individual rows show the composition of the prepared conjugate after finishing the said cycle; i.e., after finishing the procedure described in the Example 3 the product having the composition described in the first row is obtained. Then the procedure is repeated, and the product described in the second row is obtained, etc.

TABLE 5

| | S | N_{calculated} | N_{foundo} |
|---|---|---|---|
| Compound | [mmol/g] | [nmol] | [nmol] |
| H-Lys(Boc)-Nle-O-palmitoylHYA | 0.01044 | 0.157 | 0.152 |
| H-Lys(Boc)-Lys(Boc)-Nle-O- | 0.01057 | 0.314 | 0.318 |
| palmitoylHYA | | | |
| H-Lys(Boc)-Lys(Boc)-Lys(Boc)- | 0.01061 | 0.478 | 0.469 |
| Nle-O-palmitoylHYA | | | |
| H-Lys(Boc)-Lys(Boc)-Lys(Boc)- | 0.01053 | 0.631 | 0.619 |
| Lys(Boc)-Nle-O-palmitoylHYA | | | |

### Example 11

### Preparation of a Peptide Comprising 7 Lysine Units Arranged in a Dendrimer Structure—Use of the Palmitoyl HYA Fibre

1 m of H-Nle-O-palmitoylHYA prepared according to the Example 9 was placed into the 2 ml syringe reactor equipped with a frit; the fibre was repeatedly washed with DMF. A solution of 3 eq. of Fmoc-Lys(Boc)-OH, and 3 eq. of OxymaPure in 0.5 ml anhydrous DMF was prepared outside the reactor; after dissolving all components, 3 eq. of DIC were added to activate the carboxyl group of the amino acid. This solution was transferred into the reactor to the fibre. The reaction proceeded at the temperature of 18 to 23° C. for the next 20 hours. The reaction was terminated by filtering the reaction solution off. The fibre was then washed with 3×1.5 ml of DMF, 3×1.5 ml of DCM, 3×1.5 ml of IPA, 3×1.5 ml of DCM, 3×1.5 ml of DMF. Cleavage of Fmoc protecting group was performed by adding 1.5 ml of 20% solution of piperidine in DMF into the reactor to the fibre. The reaction proceeded for 5 min at the temperature of 18 to 23° C. The reaction was repeated while extending the cleavage time to 20 min. The reaction was terminated by filtering the reaction solution off. The fibre was then washed with 3×1.5 ml of DMF, 3×1.5 ml of DCM, 3×1.5 ml of IPA, 3×1.5 ml of DCM, 3×1.5 ml of DMF. This procedure was repeated 2 times.

The reaction was monitored by Fmoc-release test. The yield was determined by Fmoc-release test. The composition of the product, shown in FIG. 5, where HYA denotes palmitoyl hyaluronan, was confirmed by amino acid analysis, as presented in Table 6. It is a serial synthesis performed by the step-by-step method; however, this procedure was repeated for all the said derivatives. The individual rows show the composition of the prepared conjugate after finishing said cycle; i.e., after finishing the procedure described in the Example 3 the product having the composition described in the first row is obtained. Then the procedure is repeated, and the product described in the second row is obtained, etc.

TABLE 6

| | S | N_{calculated} | N_{found} |
|---|---|---|---|
| Compound | [mmol/g] | [nmol] | [nmol] |
| H-Lys-Nle-O-palmitoylHYA | 0.00984 | 0.16 | 0.154 |
| H-Lys₃-Nle-O-palmitoylHYA | 0.02130 | 0.49 | 0.501 |
| H-Lys₇-Nle-O-palmitoylHYA | 0.04265 | 1.12 | 1.113 |

### Example 12

### Anchoring Fmoc-Lys-NH₂ to formylHYA Fibre

1 m of formylHYA fibre (11 mg, 0.028 mmol, Mw=3.10⁵ g·mol⁻¹) was placed into a 2 ml syringe reactor equipped with a frit; the fibre was repeatedly washed with DMF. The solution of 3 eq. of Fmoc-Lys-NH₂ in 0.5 ml of anhydrous DMF was prepared outside the reactor and transferred into the reactor to the fibre. The reaction proceeded at the temperature of 18 to 23° C. for the next 20 hours. Then 3 eq. of picBH3 were added and the reaction proceeded at the temperature of 18 to 23° C. for the next 24 hours, then it was terminated by filtering the reaction solution off. The fibre was then washed with 3×1.5 ml of DMF, 3×1.5 ml of DCM, 3×1.5 ml of IPA, 3×1.5 ml of DCM, 3×1.5 ml of DEE. Cleavage of Fmoc protecting group was performed by adding 1.5 ml of 20% solution of piperidine in DMF into the reactor to the solution. The reaction proceeded for 5 min at the temperature of 18 to 23° C. The reaction was repeated while extending the cleavage time to 20 min and then it was terminated by filtering the reaction solution off. The fibre was then washed with 3×1.5 ml of DMF, 3×1.5 ml of DCM, 3×1.5 ml of IPA, 3×1.5 ml of DCM, 3×1.5 ml of DEE.

Releasing of the amino group was confirmed by performing a ninhydrin confirmation reaction (Kaiser test).

### Example 13

### Anchoring the Peptide with RGD Motif to the Fibre of Native HYA

1 m of native HYA fibre (12 mg, 0.03 mmol, Mw=3.10⁵ g·mol-) was placed into a 2 ml syringe reactor equipped with a frit; the fibre was repeatedly washed with DMF. A solution of 3 eq. of a peptide having the appropriate sequence, 3 eq. of OxymaPure in 0.3 eq. of DMAP in 0.5 ml of anhydrous DMF was prepared outside the reactor; after dissolving all the components 3 eq. of DIC were added to activate the carboxyl group of the amino acid. This solution was transferred into the reactor to the fibre. The reaction proceeded at the temperature of 18 to 23° C. for the next 20 hours. The reaction was terminated by filtering the reaction solution off. Then the fibre was washed with 3×1.5 ml of DMF, 3×1.5 ml of DCM, 3×1.5 ml of IPA, 3×1.5 ml of DCM, 3×1.5 ml of DEE.

The yield of the condensation reaction was determined by Fmoc-release test as the substitution in mmol/g. The composition of the product was confirmed by amino acid analysis as the proportion of individual amino acids. The purity of the material was determined by means of MS-HPLC after the degradation of the material. The results of the individual analyses are shown in the following Table 7:

TABLE 7

| | S | Amino acid | | | | | Purity |
|---|---|---|---|---|---|---|---|
| Compound | [mmol/g] | Ahx | Asp | Gly | Nle | Arg | [%] |
| Ac-Arg-Gly-Asp-Gly-Gly-Gly-Nle-O-HYA | — | — | 1.04 | 3.89 | 1 | 1.03 | 90 |
| H-Arg-Gly-Asp-Gly-Gly-Gly-Nle-O-HYA | 0.01802 | — | 1.01 | 3.91 | 1 | 0.97 | 82 |
| H-Gly-Gly-Gly-Arg-Gly-Asp-Nle-O-HYA | 0.01832 | — | 1.04 | 4.07 | 1 | 1.08 | 85 |
| Ac-Arg-Gly-Asp-Ahx-Ahx-Nle-O-HYA | — | 2.20 | 1.04 | 1.06 | 1 | 1.01 | 95 |
| H-Arg-Gly-Asp-Ahx-Ahx-Nle-O-HYA | 0.01843 | 2.27 | 0.93 | 1.05 | 1 | 1.00 | 90 |
| Ac-Ahx-Ahx-Arg-Gly-Asp-Nle-O-HYA | — | 2.21 | 1.03 | 1.26 | 1 | 1.01 | 86 |
| H-Ahx-Ahx-Arg-Gly-Asp-Nle-O-HYA | 0.01831 | 2.31 | 0.98 | 1.05 | 1 | 1.02 | 89 |

### Example 14

### Anchoring the Peptide Having RGD Motif to the Native HYA Nonwoven Fabric

5 squares of the side of 5 mm made of nonwoven fabric of native HYA (10.5 mg, 0.03 mmol, Mw=1.04×10⁶ g·mol⁻¹) were placed into a 2 ml syringe reactor equipped with a frit; the material was repeatedly washed with DMF. A solution of 3 eq. of a peptide having the appropriate sequence and 3 eq. of OxymaPure in 0.5 ml of anhydrous DMF was prepared outside the reactor; after dissolving all of the components, 3 eq. of DIC were added to activate the carboxyl group of the amino acid. This solution was transferred into the reactor to the material. The reaction proceeded at the temperature of 18 to 23° C. for the next 20 hours. The reaction was terminated by filtering the reaction solution off. Then the fabric was washed with 3×1.5 ml of DMF, 3×1.5 ml of DCM, 3×1.5 ml of IPA, 3×1.5 ml of DCM, 3×1.5 ml of DEE.

The yield of the condensation reaction was determined by Fmoc-release test as the substitution in mmol/g. The composition of the product was confirmed by an amino acid analysis as the proportional occurence of the individual amino acids. The purity of the material was determined by means of MS-HPLC after the degradation of the material. The results of the individual analysis are presented in the following Table 8:

TABLE 8

| | S | Amino acid | | | | | Purity |
|---|---|---|---|---|---|---|---|
| Compound | [mmol/g] | Ahx | Asp | Gly | Nle | Arg | [%] |
| H-Arg-Gly-Asp-Gly-Gly-Gly-Nle-O-HYA | 0.01802 | — | 1.04 | 3.94 | 1 | 1.01 | 85 |
| H-Gly-Gly-Gly-Arg-Gly-Asp-Nle-O-HYA | 0.01832 | — | 1.06 | 4.14 | 1 | 1.07 | 76 |
| Ac-Arg-Gly-Asp-Ahx-Ahx-Nle-O-HYA | — | 2.17 | 0.96 | 1.05 | 1 | 1.04 | 95 |
| H-Arg-Gly-Asp-Ahx-Ahx-Nle-O-HYA | 0.01843 | 2.15 | 1.01 | 1.12 | 1 | 1.06 | 88 |

### Example 15

### Anchoring the Peptide Fmoc-(VPGLG)₂-OH to a Fibre of Native HYA

1 m of native HYA fibre (12 mg, 0.03 mmol, Mw=3.10⁵ g·mol⁻¹) was placed into a 2 ml syringe reactor equipped with a frit; the fibre was repeatedly washed with DMF. A solution of 3 eq. of the peptide Fmoc-(VPGLG)₂-OH, 3 eq. of OxymaPure, and 0.3 eq. of DMAP in 0.5 ml of anhydrous DMF was prepared outside the reactor; after dissolving all of the components, 3 eq. of DIC were added to activate the carboxyl group of the amino acid. This solution was transferred into the reactor to the fibre. The reaction proceeded at the temperature of 18 to 23° C. for further 20 hours, and it was terminated by filtering the reaction solution off. Then the fibre was washed with 3×1.5 ml of DMF, 3×1.5 ml of DCM, 3×1.5 ml of IPA, 3×1.5 ml of DCM, 3×1.5 ml of DEE.

The yield of the condensation reaction was determined by Fmoc-release test as the substitution in mmol/g. The composition of the product was confirmed by an amino acid analysis as the proportional occurrence of the individual amino acids. The purity of the material was determined by means of MS-HPLC after the degradation of the material. The results of the individual analysis are shown in the following Table 9:

TABLE 9

| | S | Amino acid | | | | Purity |
|---|---|---|---|---|---|---|
| Compound | [mmol/g] | Gly | Leu | Pro | Val | [%] |
| H-(Val-Pro-Gly-Leu-Gly)₂-O-HYA | 0.01809 | 2.37 | 1.26 | 1.13 | 1 | 85 |

### Example 16

### Anchoring Dalagrin to the Fibre of Native HYA

1 m of native HYA fibre (12 mg, 0.03 mmol, Mw=3.10⁵ g·mol⁻¹) was placed into a 2 ml syringe reactor equipped with a frit; the fibre was repeatedly washed with DMF. A solution of 3 eq. of the peptide Fmoc-Dalagrin, 3 eq. of OxymaPure, and 0.3 eq. of DMAP in 0.5 ml of anhydrous DMF was prepared outside the reactor; after dissolving all of the components, 3 eq. of DIC were added to activate the carboxyl group of the amino acid. This solution was transferred into the reactor to the fibre. The reaction proceeded at the temperature of 18 to 23° ° C. for further 20 hours, and it was terminated by filtering the reaction solution off. Then the fibre was washed with 3×1.5 ml of DMF, 3×1.5 ml of DCM, 3×1.5 ml of IPA, 3×1.5 ml of DCM, 3×1.5 ml of DEE.

The yield of the condensation reaction was determined by Fmoc-release test as the substitution in mmol/g. The composition of the product was confirmed by an amino acid analysis as the proportional occurrence of the individual amino acids. The purity of the material was determined by means of MS-HPLC after the degradation of the material. The results of the individual analysis are shown in the following Table 10:

TABLE 10

| | S | Amino acid | | | | | | | Purity |
|---|---|---|---|---|---|---|---|---|---|
| Compound | [mmol/g] | ala | Arg | Gly | Nle | Leu | Phe | Tyr | [%] |
| H-Tyr-ala-Gly-Phe-Leu-Arg- | 0.01894 | 0.99 | 0.99 | 0.96 | 1 | 0.98 | 0.96 | 0.99 | 93 |
| Nle-O-HYA | | | | | | | | | |

### Example 17

### In Vitro Cell Adhesion on the Fibre Comprising RGD Motif

The fibre of native HYA comprising RGD motif, prepared according to the procedure described in Example 14, and control fibres were cut into 1 cm pieces and transferred onto a non-adhesive panel with 24 wells. Primary human fibroblasts (NHDF) were pre-marked with the fluorescent dye DiI (Exₘₐₓ/Emₘₐₓ 549/565) and inoculated, in the amount of 10⁵ cells, into the wells filled with the tested samples. Then the panel was shaken by a shaker (5 hrs/160 rpm) to keep the cells in suspension, under cultivation conditions (37° C., 5% CO₂). After 24 h of cultivation the fibres were transferred into a well with fresh cultivation medium. Then the cells were observed and detected by fluorescent microscope Nikon Ti-Eclipse with the use of TRITC filter (Exₘₐₓ/Emₘₐₓ, 549/565). The fibroblasts proliferation was monitored for 7 days of cultivation.

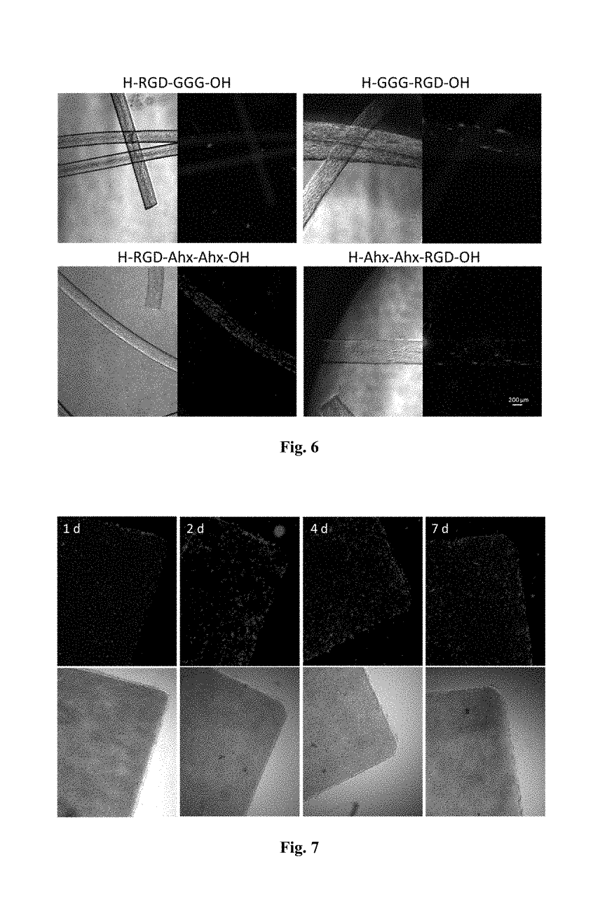

Human dermal fibroblasts adhered and proliferated during the time only on the fibre with the peptide H-Arg-Gly-Asp-Ahx-Ahx-Nle-OH bonded to HYA via 2 units of 6-aminohexanoic acid that form a firm and elastic linker and thus make the RGD peptide accessible for cell adhesion receptors. On the contrary, the peptide H-Ahx-Ahx-Arg-Gly-Asp-Nle-OH, as well as the linking of peptides H-Arg-Gly-Asp-Gly-Gly-Gly-Nle-OH and H-Gly-Gly-Gly-Arg-Gly-Asp-Nle-OH via triglycine linker, did not support the cell adhesion, as can be seen in FIG. 6. Based on these data we can assume that it is the RGD motif which supports the cell adhesion, being the minimum domain that can be recognized by cell receptors, eventually the Ahx-linker. The positive effect of Ahx-linker was then displaced by testing the fibres with the bonded peptide comprising RGD or RGD motif, where NHDF adhered exclusively on the peptide with RGD.

### Example 18

### In Vitro Cell Adhesion on a Nonwoven Fabric of Native HYA Comprising RGD Motif

Nonwoven fabric of HYA with an anchored peptide H-Arg-Gly-Asp-Ahx-Ahx-Nle-OH prepared according to the procedure described in Example 14, and a control fabric were cut into squares with a side length of 0.5 cm, and were transferred onto 24 wells plate of non-adhesive panel. Primary human fibroblasts (NHDF) were pre-marked with the fluorescent dye DiI (Exₘₐₓ/Emₘₐₓ 549/565) and inoculated, in the amount of 10⁵ cells, into the wells filled with the tested samples. Then the panel was shaken by a shaker (5 hrs/160 rpm) to keep the cells in suspension, under cultivation conditions (37° C., 5% CO₂). After 24 h of cultivation the fabric was transferred into a well with a fresh culture media. Then the cells were observed and detected by the fluorescent microscope Nikon Ti-Eclipse with the use of TRITC filter (Exₘₐₓ/Emₘₐₓ, 549/565). The fibroblasts proliferation was monitored for 7 days of cultivation, as can be seen in FIG. 7.

NHDF adhered uniformly onto the fabric surface and after 48 hours they elongated in a characteristic manner.

### INDUSTRIAL APPLICABILITY

The new medical preparation with a carrier based on hyaluronan and its derivatives can be used as a suitable drug form for treatment with the attached medical substance. This new dosage form brings an improved prolonged activity of the medical substance bound on the solid carrier of the complex system. Suitable forms of hyaluronan with the bound medical substances can be used in human and veterinary medicine.

1.-26. (canceled)

27. A method of preparing a medical preparation based on hyaluronic acid and/or palmitoyl hyaluronan or formyl hyaluronan, said method comprising binding a medical substance A comprising a terminal N-protecting group and comprising amino acids and/or peptides to a carrier N in an aprotic polar solvent, the carrier N comprising hyaluronic acid, palmitoyl hyaluronan, and/or formyl hyaluronan and having a molecular weight of from 1.5×10⁴ to 2.5×10⁶ g·mol¹, and then cleaving off the terminal N-protecting group of the medical substance Abound on the carrier in a basic environment to form a conjugate;
wherein the carrier N is in solid phase during the whole preparation, is insoluble in the reaction environment, and is biodegradable; and
wherein the conjugate has the general formula X:
A-S—N (X),
which is also represented by the formula: 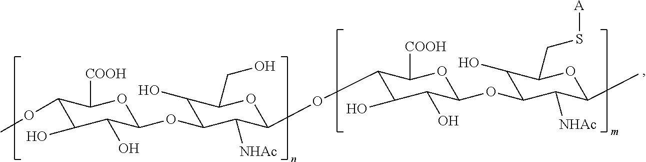
where
A is a peptide or an amino acid;
S is —O—C(═O)—, —NH—, or a linear linker based on a peptide comprising hydrophobic amino acids Xaa;
m is from 10 to 1250; and
n is from 100 to 12500.

28. The method according to claim 27, wherein the carrier N is in the form of an endless fibre, thread, textile, thin film, staple fibre, and/or nonwoven fabric.

29. The method according to claim 27, wherein the formation of the conjugate is performed at the temperature of from 20° C. to 40° C. for a time of from 2 to 48 hours.

30. The method according to claim 27, wherein the aprotic polar solvent is selected from the group consisting of N,N-dimethyl formamide, dimethyl sulphoxide, N-methyl-2-pyrrolidone, acetonitrile, dichloro methane, tetrahydrofuran, 1,4-dioxane, and combinations thereof and/or the basic environment in which the N-terminal protecting group is cleaved off is selected from the group consisting of 20% piperidine in DMF, 2% DBU in DMF, and 30% tert-butylamine in DMF.

31. The method of preparation according to claim 27, wherein binding the medical substance A to the carrier N comprises esterification; wherein the medical substance A is an amino acid or peptide comprising the terminal N-protecting group and a carboxyl group; wherein the medical substance A is first subjected to activation of the carboxyl group via a condensation agent in an aprotic polar solvent to form a reactive intermediate that is subsequently reacted with the carrier N in the presence of an organic base and a catalyst; and wherein the terminal N-protecting group of the medical substance A bound on the carrier is then cleaved off in a basic environment to form the conjugate according to the general formula X, where S is —O—C(═O)— or a linear linker based on a peptide comprising hydrophobic amino acids Xaa.

32. The method according to claim 31, wherein the condensation agent comprises N,N′-diisopropyl carbodiimide, N,N′-dicyclohexyl carbodiimide, 1-[bis(dimethylamino) methylen]-1H-1,2,3-triazolo[4,5-b] pyridinium 3-oxide hexafluoro phosphate, 1-ethyl-3-(3-dimethyl aminopropyl) carbodiimide, propylphosphonic acid anhydride, 2-(1H-benzotriazole-1-yl)-1,1,3,3-tetramethyl uronium hexafluoro phosphate, ethyl chloroformiate, benzotriazole-1-yl-oxy-tris(dimethylamino) phosphonium hexafluoro phosphate, O-(benzotriazole-1-yl)-N,N,N,N′-tetramethyl uronium tetrafluoroborate, 4-triethylamino-dicyclohexyl carbodiimide p-toluen sulfonate, 4-nitrophenol, N-hydroxy succinimide, 2,4,5-trichloro phenol, 2,3,4,5,6-pentafluoro phenol, and/or 2,3,4,5,6-pentachloro phenol.

33. The method according to claim 31, wherein the organic base is selected from the group consisting of triethylamine, pyridine, morpholine, N-methyl morpholine, N,N′-diisopropyl ethylamine, imidazole, and/or the catalyst is selected from the group consisting of ethyl(hydroxyimino) cyanoacetate, hydroxybenzo triazole, 1-hydroxy-7-azabenzotriazole, or N,N-dimethylamino pyridine.

34. The method according to claim 31, wherein the conjugate is further repeatedly reacted with the reactive intermediate which can be the same or different from the reactive intermediate of the previous step.

35. The method according to claim 31, wherein the condensation agent is N,N′-diisopropyl carbodiimide, the aprotic polar solvent is N,N-dimethyl formamide, the catalyst is a combination of ethyl (hydroxyimino) cyanoacetate and N,N-dimethylamino pyridine, and the basic environment is 20% piperidine in N,N-dimethyl formamide.

36. The method according to claim 27, wherein the amount of the medical substance A corresponds to 1 to 5 equivalents, with respect to a dimer of hyaluronic acid or a derivative thereof.

37. The method according to claim 31, wherein the amount of the condensation agent corresponds to 0.1 to 5 equivalents, with respect to a dimer of hyaluronic acid or a derivative thereof, and/or the amount of the organic base corresponds up to 10 equivalents, with respect to a dimer of hyaluronic acid or a derivative thereof, and/or the amount of the catalyst corresponds 0.1 to 5 equivalents, with respect to a dimer of hyaluronic acid or a derivative thereof.

38. The method according to claim 31, wherein 3 equivalents of ethyl(hydroxyimino) cyanoacetate as the catalyst and 0.3 equivalents of N,N-dimethylamino pyridine as the organic base are used, each with respect to a dimer of hyaluronic acid or a derivative thereof.

39. The method according to claim 31, wherein the amount of the medical substance A corresponds to 3 equivalents, the amount of the condensation agent corresponds to 3 equivalents, and the amount of the catalyst corresponds to 3 equivalents, each with respect to a dimer hyaluronic acid or a derivative thereof.

40. The method according to claim 31, wherein the amount of the medical substance A corresponds to 1 equivalent, the amount of the condensation agent corresponds to 1 equivalent, and the amount of the catalyst corresponds to 1 equivalent, with respect to a dimer of hyaluronic acid or a derivative thereof.

41. The method of preparation according to claim 27, wherein binding the medical substance A to the carrier N comprises reductive amination in the presence of a polar aprotic solvent and a reduction agent; and wherein the terminal N-protecting group of the medical substance A bound on the carrier is then cleaved off in a basic environment to form the conjugate according to the general formula X, where S is —NH—, or a linear linker based on a peptide comprising hydrophobic amino acids Xaa.

42. The method according to claim 31, wherein the conjugate is further repeatedly reacted with the medical substance A that can be the same or different from the medical substance A of the previous step.

43. The method of preparation according to claim 31, wherein the polar aprotic solvent is DMF, the reduction agent is picBH₃, and the basic environment is 20% solution of piperidine in DMF.

44. The method according to claim 27, wherein the medical substance A is bound to the carrier via a linear linker having the general formula Xaa-Ahx-Ahx-Xaa.

45. A medical preparation with a carrier based on hyaluronic acid and/or palmitoyl hyaluronan or formyl hyaluronan, the medical preparation comprising a conjugate according to the general formula:
A-S—N (X),
which is also represented by the formula: 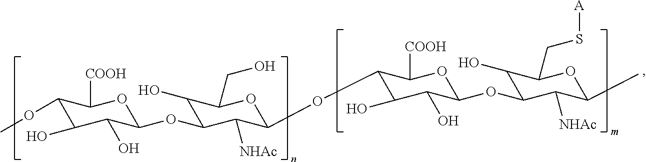
where
A is a peptide or an amino acid;
S is —O—C(═O)—, —NH—, or a linear linker based on a peptide comprising hydrophobic amino acids Xaa;
m is from 10 to 1250; and
n is from 100 to 12500.

46. The medical preparation according to claim 45 for use in medical applications as a dosage form of an active peptide or amino acid, wherein the medical preparation is formulated for administration dermally, sublingually, buccallly, or locally into an open wound.

47. The medical preparation according to claim 45, wherein the medical preparation is formulated for dermal application and comprises the peptide Dalargin as the medical substance A.

48. The medical preparation according to the claim 45, wherein the medical preparation is formulated for oral or sublingual application and comprises, as the medical substance A, a peptide selected from the group consisting of Desmopressin, Lysipressin, and Glypressin.

49. The medical preparation according to claim 45, wherein the medical preparation is formulated for buccal application and comprises, as the medical substance, a peptide selected from the group consisting of antivirotics and adjuvants, releasing factor for luteinizing and follicle-stimulating hormones, and combinations thereof.

50. The medical preparation according to claim 45, wherein the medical preparation is formulated for direct application into an open wound and comprises, as the medical substance, a peptide selected from the group consisting of Glypressin, Dalargin, and AdDP.
